(19) United States
(12) Patent Application Publication
(10) Pub. No.: US 2009/0086891 A1
(43) Pub. Date: Apr. 2, 2009

(54) BREAST IMAGE DISPLAY APPARATUS AND ITS PROGRAM

(75) Inventor: Akio OFUJI, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/240,309

(22) Filed: Sep. 29, 2008

(30) Foreign Application Priority Data
Sep. 27, 2007 (JP) 2007-250563

(51) Int. Cl. A61B 6/04

(52) U.S. Cl. 378/37

(57) ABSTRACT

The mammary-gland content rate of each of mammograms is calculated. The mammograms are sorted in a certain order based on the mammary-gland content rate of each of the mammograms. Further, the mammograms are displayed according to the order, in which the mammograms have been sorted.

### BACKGROUND OF THE INVENTION

1. Field of the Invention

The present invention relates to an image diagnosis assistance apparatus. Specifically, the present invention relates to a breast image display apparatus that changes its method for displaying mammograms (radiographic images of a breast) based on the amounts of mammary glands included in the mammograms and to a program related to the breast image display apparatus.

2. Description of the Related Art

Conventionally, in examination of breast cancer, a combination of palpation and examination using mammograms (mammography), obtained by imaging a breast, can greatly increase the rate of detection of early cancer, compared with examination only by palpation. Therefore, medical examination using mammography apparatuses (radiography apparatuses specialized in examination of breasts) has been performed.

When mammograms obtained by imaging are diagnosed, a plurality of mammograms are arranged and displayed on a high-resolution display device. Then, diagnosis is performed by comparing the displayed mammograms with each other. Since abnormal shadows, such as breast cancer, tend to be present in mammary gland areas, a mammary gland region is observed in diagnosis. However, the condition of distribution of mammary glands greatly differs in individuals and the abnormal shadows look different according to the condition of the distribution of the mammary glands. In mammograms in which the degree of distribution of mammary glands is low, a difference in density between abnormal shadows and normal tissue is large. Therefore, the abnormal shadows clearly appear in the mammograms. However, in mammograms in which the degree of distribution of mammary glands is high, a difference in density between abnormal shadows and normal tissue is small. Therefore, it is difficult detect the abnormal shadows. Hence, special attention is needed in diagnosis of such mammograms.

Further, when doctors (physicians, surgeons, radiologists, oncologists or the like) read mammograms, they observe the mammograms and classify the breasts into types based on the condition of distribution of mammary glands in the mammograms. However, the results of classification differ among the doctors. Further, it is difficult even for the same doctor to replicate his/her classification. Therefore, a diagnosis assistance apparatus that can replicate classification by quantitatively analyzing mammograms has been proposed. In the diagnosis assistance apparatus, the mammograms are classified based on the condition of distribution of mammary glands by a computer. When doctors read (diagnose) the mammograms, the result of classification by the computer is displayed. Therefore, the doctors can detect abnormal shadows with reference to the displayed result of classification, thereby reducing an error due to oversight in detection of abnormal shadows that may be present in mammary glands. Further, image processing (for example, image contrast, LUT (look-up table), density, the degree of frequency emphasis) is performed based on the result of classification to detect abnormal shadows (for example, Japanese Unexamined Patent Publication 2002-125961 or the like).

When doctors diagnose patients (radiographs), if the radiographs are mammograms, the doctors pay attention to mammary gland regions of the mammograms. In such a case, the doctors need to consider the condition of distribution of the mammary glands, as described above. However, when they need to observe a mammogram in which a large amount of mammary glands is present immediately after observing a mammogram in which a small amount of mammary glands is present, the image of the previous mammogram, namely the mammogram with the small amount of mammary glands, remains in their consciousness. Therefore, there are cases in which correct diagnosis is not performed by the influence of the remained image. Even if the result of classification is displayed as disclosed in Japanese Unexamined Patent Publication 2002-125961, diagnosis is often influenced by the previous image, which was observed immediately before the current image.

### SUMMARY OF THE INVENTION

In view of the foregoing circumstances, it is an object of the present invention to provide a breast image display apparatus that displays mammograms in such a manner that correct diagnosis that is not influenced by a difference in the amounts of mammary glands of breasts can be performed.

A breast image display apparatus according to the present invention is a breast image display apparatus comprising:

an image storage means for storing a plurality of mammograms;

a mammary-gland content rate calculation means for calculating the mammary-gland content rate of each of the plurality of mammograms;

a sort means for sorting the plurality of mammograms in a certain order based on the mammary-gland content rates thereof, obtained by the mammary-gland content rate calculation means; and

a first breast image display means for displaying the plurality of mammograms according to the order, in which the plurality of mammograms have been sorted by the sort means.

A breast image display program of the present invention is a breast image display program for causing a computer to function as:

a mammary-gland content rate calculation means for calculating the mammary-gland content rate of each of a plurality of mammograms;

a sort means for sorting the plurality of mammograms in a certain order based on the mammary-gland content rates thereof, obtained by the mammary-gland content rate calculation means; and

a first breast image display means for displaying the plurality of mammograms according to the order, in which the plurality of mammograms have been sorted by the sort means.

The term “mammary-gland content rate” refers to the ratio of the area of a portion that is judged as a mammary gland region with respect to the area of a breast region (including a fat region and the mammary gland region) that has been detected from a mammogram.

The expression “sorting . . . in a certain order based on the mammary-gland content rates” refers to sorting them in ascending order or in descending order based on the mammary-gland content rates thereof, obtained by calculation.

The expression “displaying the plurality of mammograms according to the order, in which the plurality of mammograms have been sorted” may refer to displaying the plurality of mammograms, one by one, in ascending order or in descending order based on the mammary-gland content rates thereof by switching the plurality of mammograms. Alternatively, the expression may refer to displaying a plurality of mammograms at the same time in the same display, arranging them in ascending order or in descending order based on the mammary-gland content rates thereof.

Another breast image display apparatus according to the present invention is a breast image display apparatus comprising:

an image storage means for storing a plurality of mammograms;

a mammary-gland content rate calculation means for calculating the mammary-gland content rate of each of the plurality of mammograms;

a classification means for classifying the plurality of mammograms into a plurality of breast types based on the mammary-gland content rates thereof, obtained by the mammary-gland content rate calculation means; and

a second breast image display means for displaying the plurality of mammograms separately based on their breast types, into which the plurality of mammograms have been classified by the classification means.

Another breast image display program according to the present invention is a breast image display program for causing a computer to function as:

a mammary-gland content rate calculation means for calculating the mammary-gland content rate of each of a plurality of mammograms;

a classification means for classifying the plurality of mammograms into a plurality of breast types based on the mammary-gland content rates thereof, obtained by the mammary-gland content rate calculation means; and

a second breast image display means for displaying the plurality of mammograms separately based on their breast types, into which the plurality of mammograms have been classified by the classification means.

The expression “classifying the plurality of mammograms into a plurality of breast types based on the mammary-gland content rates thereof” refers to classifying the plurality of mammograms based on their mammary-gland content rates in such a manner that mammograms of similar breast types are grouped (classified) into the same group based on a predetermined standard.

The expression “displaying the plurality of mammograms separately based on their breast types, into which the plurality of mammograms have been classified” may refer to displaying a plurality of mammograms of the same breast type in any order thereamong when the mammograms are displayed at the same time in the same display. When the mammograms are displayed one by one by switching, the expression may refer to displaying mammograms of the same breast type consecutively. As long as the mammograms of the same breast type are displayed consecutively, they may be displayed in any order thereamong.

Further, it is desirable that the plurality of breast types are a fatty type, a mammary-gland-scattered type, a heterogeneously-dense type and a dense type.

Further, it is desirable that the second breast image display means displays the breast types, into which the plurality of mammograms have been classified by the classification means, together with the plurality of mammograms.

Specifically, the expression “displays the breast types, into which the plurality of mammograms have been classified by the classification means, together with the plurality of mammograms” refers to displaying the names of the breast types, into which the plurality of mammograms have been classified, together with the plurality of mammograms. Alternatively, marks corresponding to the breast types may be displayed instead of the names of the breast types.

According to the present invention, mammograms are arranged and displayed in a certain order based on the mammary-gland content rates of the mammograms. Therefore, diagnosis can be performed at high accuracy with little influence of the mammary-gland content rate of the previous mammogram, which was diagnosed immediately before the current mammogram.

Further, the plurality of mammograms are classified into the plurality of breast types based on the mammary-gland content rates of the mammograms and displayed separately for each of the classified breast types, being divided into groups. Therefore, it is possible to observe a group of similar mammograms together. Further, when a mammogram is diagnosed, it is possible to reduce influence of the mammary-gland content rates of the other mammograms on diagnosis, thereby improving the accuracy of diagnosis.

Further, since the breast types are classified into representative four breast types, namely, a fatty type, a mammary-gland-scattered type, a heterogeneously-dense type and a dense type, it is possible to classify the breast types in an appropriate manner for diagnosis.

Further, since the classified breast types are displayed together with the mammograms, doctors (radiologists or the like) who diagnose patients by reading images (mammograms) can be conscious of the breast types to which the breasts in the mammograms have been classified.

Note that the program of the present invention may be provided being recorded on a computer readable medium. Those who are skilled in the art would know that computer readable media are not limited to any specific type of device, and include, but are not limited to: floppy disks, CD's, RAM's, ROM's, hard disks, magnetic tapes, and internet downloads, in which computer instructions can be stored and/or transmitted. Transmission of the computer instructions through a network or through wireless transmission means is also within the scope of this invention. Additionally, computer instructions include, but are not limited to: source, object and executable code, and can be in any language including higher level languages, assembly language, and machine language.

### BRIEF DESCRIPTION OF THE DRAWINGS

FIG. 1 is a schematic diagram illustrating the configuration of a medical information system;

FIG. 2 is a schematic diagram illustrating the configuration of a mammography apparatus;

FIG. 3 is schematic diagram illustrating the configuration of a breast image display apparatus according to a first embodiment of the present invention;

FIG. 4 is a diagram explaining regions in a mammogram;

FIG. 5 is a flowchart showing the flow of processing in diagnosis of mammograms;

FIG. 6 is a schematic diagram illustrating the configuration of a breast image display apparatus according to a second embodiment of the present invention;

FIG. 7 is a diagram illustrating an example in which mammograms are displayed, one by one, in an order based on mammary-gland content rates thereof by switching the mammograms;

FIG. 8 is a diagram illustrating an example in which a plurality of mammograms are displayed in an order based on mammary-gland content rates thereof;

FIG. 9 is a diagram illustrating an example in which a plurality of mammograms are classified into breast types and separately displayed for each of the breast types; and

FIG. 10 is a diagram illustrating an example in which mammograms are classified into breast types and displayed, one by one, by switching.

### DESCRIPTION OF THE PREFERRED EMBODIMENTS

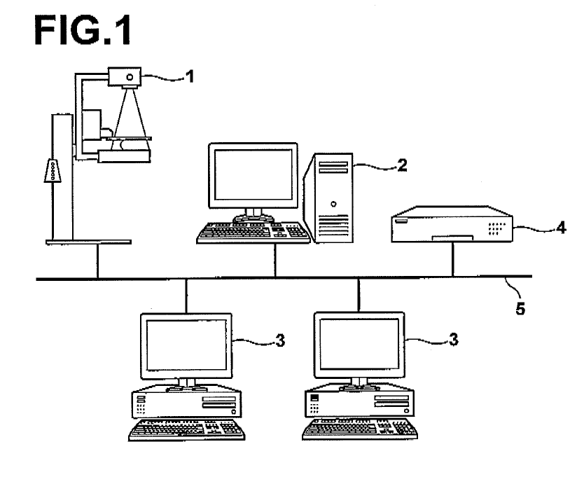

FIG. 1 is a schematic diagram illustrating the configuration of a medical information system according to a first embodiment, into which a breast image display apparatus of the present invention has been introduced. As illustrated in FIG. 1, this system includes an X-ray radiography apparatus specialized in radiography of a breast (hereinafter, referred to a mammography apparatus) 1, a workstation (QA-WS) 2 for checking image quality, a workstation 3 for image-reading (breast image display apparatus) and an image management server 4, which are connected to each other through a network 5 in such a manner that they can communicate with each other.

The network 5 is a local area network that connects various kinds of apparatuses within a hospital. Mammograms obtained by the mammography apparatus 1 are sent to or received from the apparatuses. When a workstation 3 for image-reading is installed also at another hospital or a clinic, the network 5 may be configured by connecting the local area networks of these hospitals to each other via the Internet or leased lines. Further, it is desirable that the network 5 is an optical network or the like, which can realize high-speed transfer of images.

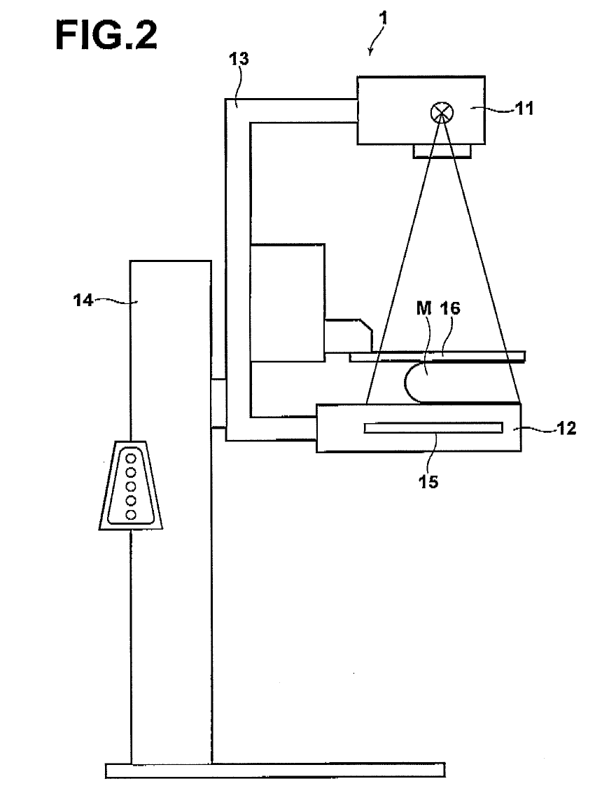

As illustrated in FIG. 2, the mammography apparatus 1 includes a radiation irradiation unit 11 for outputting radiation, an imaging table 12, on which a breast is placed, an arm 13 and a base 14 to which the arm 13 is attached. The arm 13 connects the radiation irradiation unit 11 and the imaging table 12 in such a manner that they face each other. Further, a compression plate (a press plate or a pressure plate) 16 is attached to the arm 13. The compression plate 16 is provided between the radiation irradiation unit 11 and the imaging table 12. The compression plate 16 presses breast M onto the imaging table 12 from the upper side of the imaging table 12 to compress the breast M.

Further, an image recording medium 15, such as a flat panel detector, is housed in the imaging table 12. Radiation that has been output from the radiation irradiation unit 11 passes through the breast on the imaging table 12, and a radiographic image of the breast (a mammogram) is recorded in the image recording medium 15. The breast M is a three-dimensional object and has a substantial thickness. Therefore, if the breast M is imaged without being compressed, tumors may not be clearly imaged in some cases because mammary glands, fat, blood vessels and the like block the tumors or the like. Therefore, radiation is output from the radiation irradiation unit 11 after the breast is compressed from the upper side thereof by the compression plate 16. Then, the radiation that has passed through the compressed breast M is recorded in the image recording medium 15.

Further, the mammography apparatus 1 includes a sending/receiving unit (not illustrated) that is connected to the network 5. The sending/receiving unit sends the mammogram recorded in the flat panel detector 15 to the workstation 2 for checking image quality after attaching attribute information defined in DICOM standard to the mammogram. Attribute information defined in an original standard of the manufacturer of modality or the like may be used.

The QA-WS 2 includes a general-purpose computer (for example, a workstation or the like), one or two high-resolution displays and an input device, such as a keyboard and a mouse. A software program for assisting (supporting) operations of test technologists is installed in the computer. The QA-WS 2 performs standardization processing (EDRprocessing) and processing for adjusting the quality of mammograms on the mammogram received from the mammography apparatus 1 by a function that is realized by executing the software program. Further, the QA-WS 2 displays the processed mammogram and the content of the attribute information on the display and prompts the test technologist to check the mammogram. After the mammogram is checked by the test technologist, the mammogram is transferred to the image management server 4 through the network 5.

The image management server 4 is a general-purpose computer that has relatively high processing performance, and a software program that can provide a function of a database management system (DataBase Management System: DBMS) is installed in the computer. Further, the image management server 4 includes a high capacity storage. The storage may be a high-capacity hard disk that is connected to the image management server 4 through a data bus. Alternatively, the storage may be a disk apparatus connected to the network 5.

In the image management server 4, the mammogram and the attribute information are recorded, being correlated with each other. The attribute information includes, for example, an image ID for distinguishing individual mammograms from each other (identifying or classifying individual mammograms), a patient ID for distinguishing subjects (subjects of radiography) from each other, a test ID (an examination ID) for distinguishing examinations from each other, a unique ID (UID) assigned to each mammogram, an examination date on which the mammogram was generated, examination time, the kind of modality used in examination for obtaining the mammogram, patient information, such as the name and the age of the patient, and the like. The attribute information is managed as XML or SGML data, for example.

Further, when the image management server 4 receives a browse request (a view request, a search-for-reference request or the like) from the workstation 3 for image-reading via the network 5, the image management server 4 retrieves a requested mammogram and sends the retrieved mammogram to the workstation 3 for image-reading, which has sent the request.

The workstation 3 for image-reading is used, by doctors who diagnose patients by reading images, to read mammograms and to prepare reports on the results of image-reading. The workstation 3 for image-reading includes a processing apparatus, one or two high-resolution displays and an input device, such as a keyboard and a mouse. The workstation 3 for image-reading sends a browse request to the image management server 4 to obtain a mammogram P. Then, the workstation 3 for image-reading receives the requested mammogram P from the image management server 4 and displays the received mammogram P. A breast image display program of the present invention is installed in the workstation 3 for image-reading to make the workstation 3 for image-reading function as a breast image display apparatus.

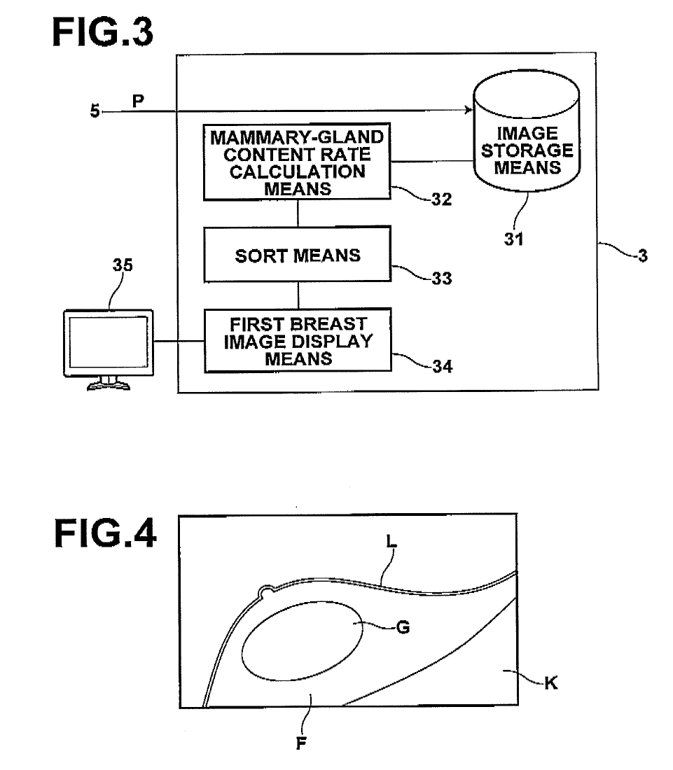

As illustrated in FIG. 3, the workstation 3 for image-reading (breast image display apparatus) includes an image storage means 31, a mammary-gland content rate calculation means 32, a sort means 33 and a first breast image display means 34. The image storage means 31 sends a browse request to the image management server 4 and obtains mammograms P that are necessary to perform image-reading. The image storage means 31 obtains and stores a plurality of mammograms P. The mammary-gland content rate calculation means 32 calculates the mammary-gland content rate of each of the mammograms P. The sort means 33 sorts the mammograms P in an order based on the mammary-gland content rates thereof. The first breast image display means 34 displays the mammograms P on the screen of a display device 35 according the sorted order.

The mammary-gland content rate calculation means 32 calculates mammary-gland content rates. First, the mammary-gland content rate calculation means 32 detects a skin line in a mammogram P to calculate the mammary-gland content rate. Then, the mammary-gland content rate calculation means 32 divides the breast region defined by the skin line into a pectoral muscle region, a mammary gland region and a fat region. For example, K-means method, which is a typical clustering method, is used to segment the breast region into regions (skin line L, pectoral muscle region K, fat region F and mammary gland region G) as illustrated in FIG. 4. Then, correspondence between the four regions and the skin line L, the pectoral muscle region K, the fat region F and the mammary gland region G is identified based on the positional relationship among the four regions and the relationship among the average density values of the four regions. Specifically, for example, a region that is located at the highest position among the four regions and that has the highest average density value is regarded as a skin line and the breast region is divided into the regions.

Next, the mammary-gland content rate of each of the mammograms P is calculated. When the area (the number of pixels) of a region that is identified as mammary glands by segmentation is Sm and the area of a region that is identified as fat is Sf, the mammary-gland content rate is obtained by using the following equation (1):

Mammary-Gland Content Rate (%)=Sm/(Sm+Sf)×100 (1).

The sort means 33 sorts the mammograms based on the mammary-gland content rates. The sort means 33 sorts the image list of the mammograms by using sort algorithm, such as quick sort and bubble sort. Users can select the sort order of the image list from the ascending order based on mammary-gland content rates and the descending order based on mammary-gland content rates.

The first breast image display means 34 displays the mammograms P according to the order sorted by the sort means 33.

Software that operates in the aforementioned devices, apparatuses or equipment may be installed therein from a recording medium, such as a CD-ROM. Alternatively, the software may be downloaded from a server connected to a network, such as the Internet, and installed.

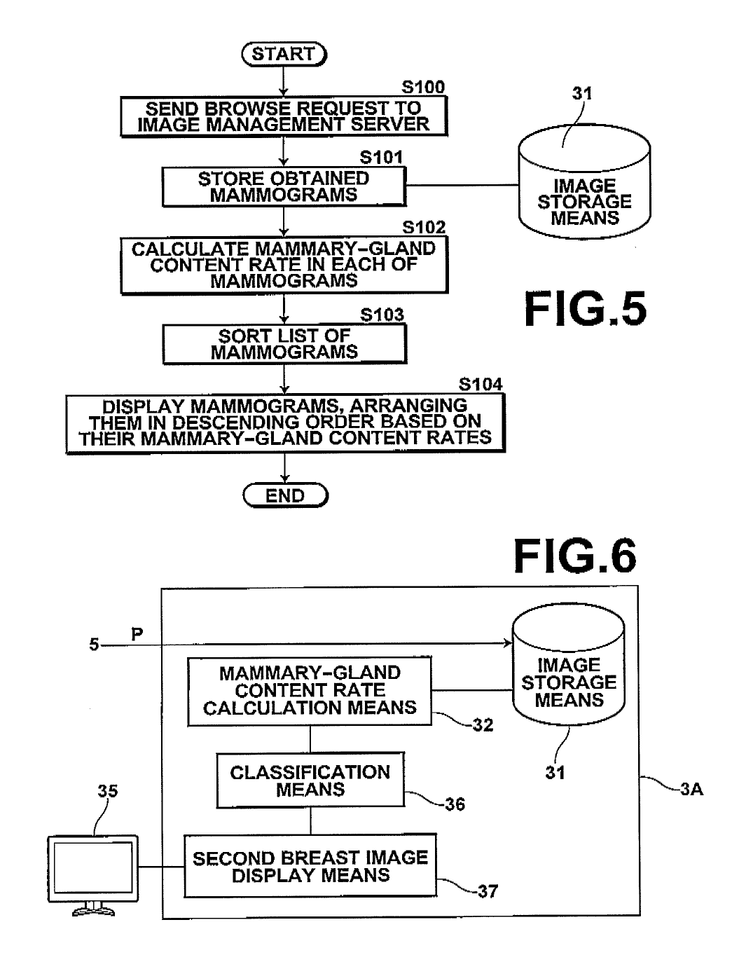

Next, the flow of processing for diagnosing mammograms by using the medial information system of the present embodiment will be described with reference to the flowchart illustrated in FIG. 5.

When a user, such as a doctor who is specialized in reading of images, inputs a browse request to view mammograms P that are targets of comparative image-reading at the workstation 3 for image-reading, the browse request is sent from the workstation 3 for image-reading to the image management server 4 (step S100). Then, a plurality of mammograms P that are necessary for comparative image-reading are obtained. The obtained mammograms P are stored in the image storage means 31 (step S101).

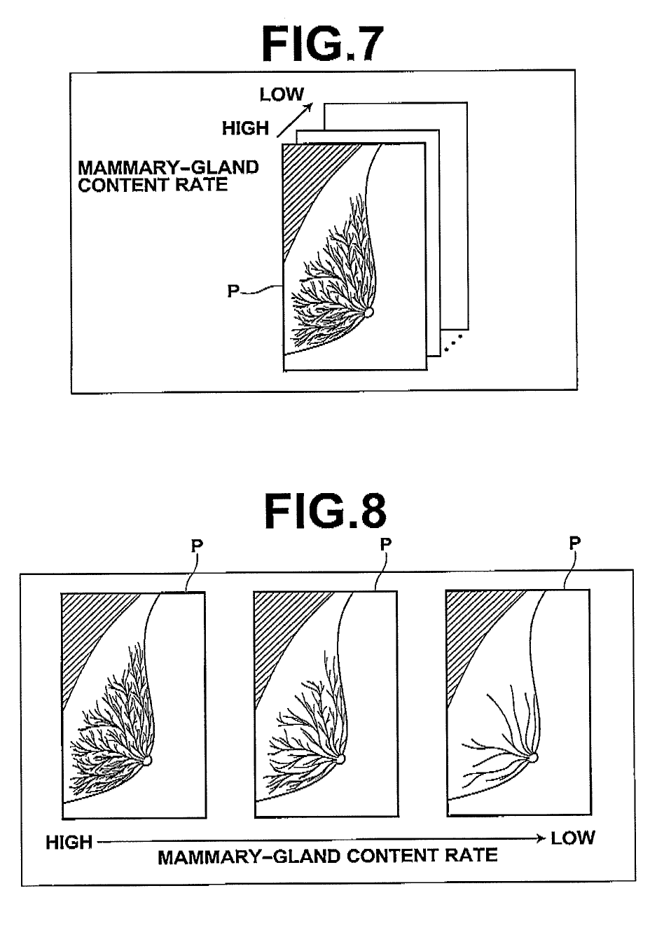

At the workstation 3 for image-reading, the mammary-gland content rate calculation means 32 calculates the mammary-gland content rate of each of the mammograms P stored in the image storage means 31 (step S102). The sort means 33 sorts the list of the mammograms P in ascending order based on the mammary-gland content rates (step S103). Then, the first breast image display means 34 displays the mammograms P on the display device based on the list that has been sorted. As illustrated in FIG. 7, the mammograms P are sequentially displayed, one by one, on the display device of the workstation 3 for image-reading. The mammograms P are displayed in descending order (or in ascending order) based on the mammary-gland content rates by switching the mammograms P (step S104) Alternatively, as illustrated in FIG. 8, a plurality of mammograms P may be displayed at the same time on the display in ascending order or in descending order based on the mammary-gland content rates.

The mammograms are arranged in an order based on the mammary-gland content rates thereof and displayed as described above. Further, image-reading is performed on the mammograms that are displayed in such a manner. Therefore, it is possible to reduce influence of the mammary-gland content rate of the previous mammogram, which was diagnosed immediately before the current mammogram, on diagnosis of the current mammogram. Hence, it is possible to improve the accuracy of diagnosis.

Next, a second embodiment of the medical information system will be described. The same signs as those used in the first embodiment will be used to represent elements corresponding to those in the first embodiment, and the detailed description of such elements will be omitted. In the second embodiment, only different elements will be described. The first embodiment was described with regard to a case in which the mammograms are sorted based on the mammary-gland content rates thereof and displayed. The second embodiment will be described with regard to a case in which the mammograms are classified into a plurality of breast types based on the mammary-gland content rates and displayed separately based on the breast types.

The configuration of the medial information system in the second embodiment is substantially the same as that of the first embodiment. Therefore, the detailed description of the configuration will be omitted. In the second embodiment, the configuration of the workstation 3 for image-reading (breast image display apparatus) differs from that of the first embodiment.

As illustrated in FIG. 6, in a workstation 3A for image-reading (breast image display apparatus) of the second embodiment, the sort means 33 provided in the first embodiment is replaced by a classification means 36. The classification means 36 classifies the plurality of mammograms P into a plurality of breast types based on the mammary-gland content rates of the mammograms. Further, the first breast image display means 34 provided in the first embodiment is replaced by a second breast image display means 37. The second breast image display means 37 displays the mammograms P separately on the display of the display device 35 based on their breast types, into which the mammograms P have been classified.

The classification means 36 classifies the mammograms P into four types based on the mammary-gland content rates of the mammograms P. Generally, the breast types are classified into “dense”, “heterogeneously dense”, “mammary-gland-scattered” and “fatty” in descending order based on the mammary-gland densities thereof. It is possible to classify the mammograms P into the four breast types by applying three threshold values for classification that have been obtained empirically. For example, if the three threshold values are 60%, 40% and 20%, the mammograms P are classified as follows:

mammary-gland content rate at 60% or higher: classified into “dense”;

mammary-gland content rate at 40% or higher and less than 60%: classified into “heterogeneously dense”;

mammary-gland content rate at 20% or higher and less than 40%: classified into “mammary-gland-scattered”; and

mammary-gland content rate at less than 20%: classified into “fatty”.

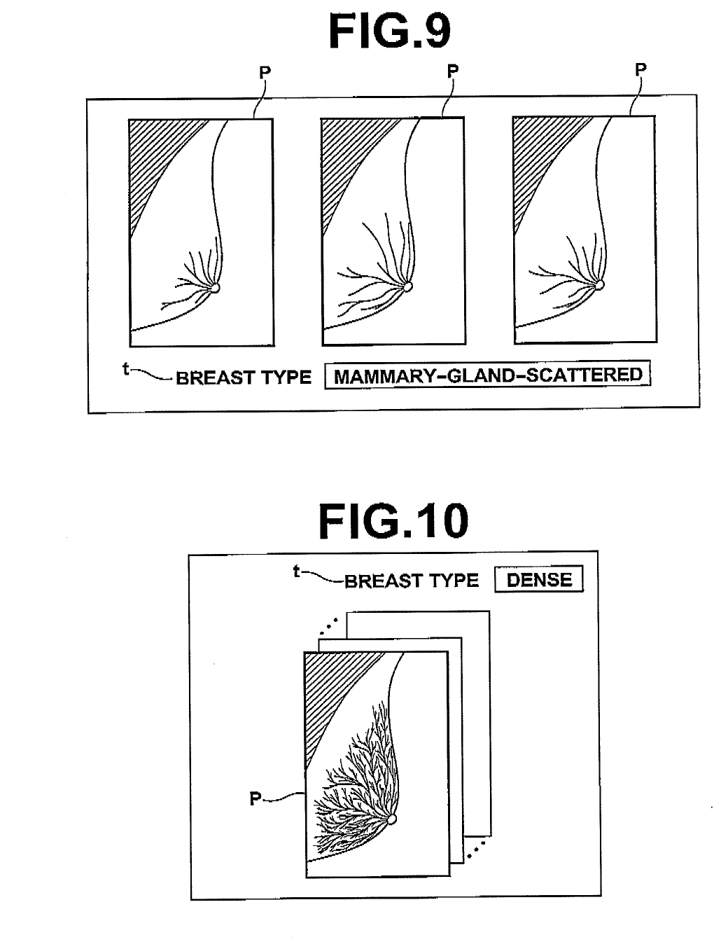

The second breast image display means 37 displays the mammograms P separately for each of the classified breast types. Specifically, as illustrated in FIG. 9, mammograms P that belong to the same breast type t are displayed at the same time on the same display. Mammograms P that belong to a different breast type t are displayed in another display. At this time, as long as the mammograms P of the same breast type t are displayed at the same time on the same display, the order of arrangement thereof may be in any order thereamong, regardless of the mammary-gland content rates. Alternatively, when the mammograms P are displayed, one by one, by switching as illustrated in FIG. 10, the mammograms P are displayed in such a manner that the mammograms P of the same breast type are displayed consecutively. After all of the mammograms P of one breast type are displayed, mammograms P of a different breast type may be displayed. At this time, the mammograms P of the same breast type t may continue to be displayed until an instruction for displaying mammograms of a different breast type t is input. When an instruction for switching the displayed mammograms to mammograms of a different breast type is input, the mammograms P of the different breast type may be displayed. Further, the mammograms of the same breast type may be displayed in any order thereamong.

Further, in both of the case of displaying the mammograms P of the same breast type t at the same time on the same display and the case of displaying the mammograms P, one by one, by switching, the name of the breast type t to which the currently-displayed mammogram or mammograms P are classified may be displayed next to the mammogram or mammograms P in the display, as illustrated in FIGS. 9 and 10. If the name of the breast type t of the mammogram P is displayed in such a manner, the doctor can diagnose the mammogram, being conscious of the breast type t of the mammogram P. Therefore, it is possible to improve the accuracy of diagnosis. Alternatively, a mark or the like corresponding to the breast type may be displayed instead of the name of the breast type t.

The flow of diagnosing mammograms by using the medical information system of the second embodiment is substantially the same as that of the first embodiment. Therefore, the explanation of the flow of processing in the second embodiment will be omitted.

As described above, the mammograms are classified into breast types and displayed separately for each of the breast types. Since image-reading is performed on the mammograms that are displayed in such a manner, diagnosis of abnormal shadows is less influenced by the mammary-gland content rates of other mammograms. Hence, it is possible to improve the accuracy of diagnosis.

What is claimed is:

1. A breast image display apparatus comprising:
an image storage means for storing a plurality of mammograms;
a mammary-gland content rate calculation means for calculating the mammary-gland content rate of each of the plurality of mammograms;
a sort means for sorting the plurality of mammograms in a certain order based on the mammary-gland content rates thereof, obtained by the mammary-gland content rate calculation means; and
a first breast image display means for displaying the plurality of mammograms according to the order, in which the plurality of mammograms have been sorted by the sort means.

2. A breast image display apparatus comprising:
an image storage means for storing a plurality of mammograms;
a mammary-gland content rate calculation means for calculating the mammary-gland content rate of each of the plurality of mammograms;
a classification means for classifying the plurality of mammograms into a plurality of breast types based on the mammary-gland content rates thereof, obtained by the mammary-gland content rate calculation means; and
a second breast image display means for displaying the plurality of mammograms separately based on their breast types, into which the plurality of mammograms have been classified by the classification means.

3. A breast image display apparatus, as defined in claim 2, wherein the plurality of breast types are a fatty type, a mammary-gland-scattered type, a heterogeneously-dense type and a dense type.

4. A breast image display apparatus, as defined in claim 2, wherein the second breast image display means displays the breast types, into which the plurality of mammograms have been classified by the classification means, together with the plurality of mammograms.

5. A breast image display apparatus, as defined in claim 3, wherein the second breast image display means displays the breast types, into which the plurality of mammograms have been classified by the classification means, together with the plurality of mammograms.

6. A breast image display program for causing a computer to function as:
a mammary-gland content rate calculation means for calculating the mammary-gland content rate of each of a plurality of mammograms;
a sort means for sorting the plurality of mammograms in a certain order based on the mammary-gland content rates thereof, obtained by the mammary-gland content rate calculation means; and
a first breast image display means for displaying the plurality of mammograms according to the order, in which the plurality of mammograms have been sorted by the sort means.

7. A breast image display program for causing a computer to function as:
a mammary-gland content rate calculation means for calculating the mammary-gland content rate of each of a plurality of mammograms;
a classification means for classifying the plurality of mammograms into a plurality of breast types based on the mammary-gland content rates thereof, obtained by the mammary-gland content rate calculation means; and
a second breast image display means for displaying the plurality of mammograms separately based on their breast types, into which the plurality of mammograms have been classified by the classification means.
